SEQUENCE LISTING (28 sequences)

SEQ ID NO: 1 - DNA, 9807 bp, Artificial Sequence
  Synthesized
atggtgtcaattgcccgaaggtacagtattagtttgaattgggcaacaacaacaaaaacgtgcgttctctaatactcacttctctagaaaacaacaaatatgataacgtaacctcctttgcttcttttcttactgatcaagaccactcgtaactaataaagatgatagatcttttatataaaaaattgtattcgtcaatgctcttttatgaaaacaatagttcaactacccatacttgcatctattagctatctagtattctcatattattacaaacattatgcagattggagataattaccaacatttctatataaacaatccgaatttggtgattataaatacttaattaccattctggaagtaacaatataacaggtagtatctatgttattttggcatgaaaggaaatgatcttaacttctcaaagtatatttcccacttttctcaagaagatcattaataaactaatacctcttcttggacagacatttttgagttatcttgtttttgtttcggtcactagttaagaaaagagaagaaaactcaacataccattttatcacgttatagccatgttcgaattgaaaactaaccacctaaaactagaggatatgatttcttatctattttttggtttcattttaagcaggaccaaaacctaaatggccggttcctccaaaactagttatattttttttttgtataattgcaaattcataaagatgaaaagagctgcattcttgggtatatacatgttaacatatctttctttagatacagtggtcctcctatgcatcacatgcaatatcgggtaaggtcaaaaagatattgaattgtccacaacacataagaagttaagatgtcactcgtggacattaacgtcgtagagtacccatatattcccatggaaattattggtattcgtaactcttggatatttttaaacgttgttaatactttaataaaatatattatattgtctaccgaatcaaacacagctgtatcaacaaaaataatttgaaacttgtataaatagatgaagtggatatgagaggaatgtaagccttatagaagagaaaaaaaatatgaattttactggctattctcgatttttaatcgtctttgtagctcttgtaggtgctcttgttcttccctcgaaaatgaggaggaactgcaacctggagctgaggctgctgccgagcagctacccgagcgacagccacgacatgatggaggagaggatcgagagcccgaagacccagcagcagcagctgaccatcttctacaacggcagggtgtgcgtgagcgacgtgaccgagctgcaggccaaggccatcctgatgctggccaacagggagagggacgagaggatgaagagcccgaccggctgggagccggtgagcccgaccctgaagagccaggtgaagtgcccgaacaccgccctgagcatgaagaggagcctgcagaggttcctgcagaagaggaagaccaggatccaggccaccagcccgtaccactacccctacgacgtgcccgactacgcctgatgatctatgcattttctttctcaaaaatgctttcattaattttctgtatctctgtcttcttgataaaatgaataaaacaaatgctcctttgtctcctatattaatctcaaacagataaagctgaaaatcccaaaaacctcttagtaattcaaaaaaaaaaaaaatagctgaaaagcaaatctctatttgatcaattaattcgaacgctcaatagtatgtaagaagtcatttacacagtacctcggtaagtgtttgagggttaaatatttgttgaatttttaacatcacactaatttagagtataatctaaattataaggtatttctctctggttatattttgttgctgcttggacgattttgtgaatgatggtagaaaaatataaagatgagaattatcaatgttttcgacgggttttcgacggacgtccgacgggttttggcgacgaatttccgacggaaattttgaatatttattttttaaaataaaaaaatgcacggctatgtgaccgtgcggcgaattgaccgagtagctacttgaccgagtgtatttcgttggaattccgtcggaaaagtgcagaatattctgatgaatattctagggatcaaaacggatatattttggtcggaaatccgtcgaaacatttggtcggatttaatttggtcggatttaatttggtcggagtttcgttggaaatgactgatgaattgtcgatcatttagaattcgtcagaaaagttcgacgacattttgacggaaaacatgtcgtcagaaaaccgtcggaataccattacgaaaccgtcgaaaattttcgaccgtttatttatcgtcgaaaagtctgtcgaaaagggttttgttctcttgtagtgtaccacaacttggtttgcaacactatatatactttttgatatataaaaattctattaaactacttaaatatttcgtcatgaggttatatgcattagaaaaaaatatataacaatattgttggaagactggacaattatcgttgaaactatagctatcaccaagatacaactttttgtggaaaatcttggtggcaattagaaactgttcccaatctcgggcatgaaaatcgttattcaaatatttgtcaacaactagtgaatagtgatacagctatatatgtttgctaatttattgaattatttaatgttacgactttacgtaacaattatttaacgtctattcttgtgtacctcacatttcttatcgtcatgtctcatctcttatattattcacagtacacccatctcttctcgctcactgtggaacctgttgtcaattactcgttttgcattttattggttttcccaattactctatcaattatttatcaaaaaaaaaaaaatgaaacatttactctatatattatttccgcaaacacaaattatactcacatcaacatattcaatacatttttctagtaatgtagaacaactttacagtattctccaaaacgaaactctaattcaaaatttacaagcagataagccaaagataatagaacaacaaaacgccaaattctagttaagcacacaatctcaacgtgcactaaaaacgagtggtgtaagtgaaaaatatcgtcgattataaacattatgggaccagtagcatttgttgcaccaatcgaaaacagacaagcacacatatctcctcatttctcatctggcttcttaatcatttctcataaccccacctcattataaataccaccctttgcgtcacacatataaacatcacaaactaaacaataaaccataccataaaaaacatgcctccacaaaaagaaaaccatagaacattgaacaaaatgaagactaatctttttctctttctcatcttttcacttctcctatcattatcctcgatgaggaggaactgcaacctcgagctcaggctcctcccatcctcctacccatccgactcccacgacatgatggaggagaggatcgagtccccaaagacccaacaacaacaactcaccatcttctacaacggcagggtgtgcgtgtccgacgtgaccgagctccaagccaaggccatcctcatgctcgccaacagggagagggacgagaggatgaagtccccaaccggctgggagccagtgtccccaaccctcaagtcccaagtgaagtgcccaaacaccgccctctccatgaagaggtccctccaaaggttcctccaaaagaggaagaccaggatccaagccacctccccataccacgaacagaaactgatctctgaagaagatctgtgatgagaatctatcttcatcgcattgtccccttgtatacacttcatatctatgtcgtttcgtttatctttgtagccgttttggcaccgctgcataaataaaatgtctatcctatcgtaacttaataagtacaaagacttcgtactaaatgttgtttttctttaaaggggtcattatataagtggccatgaaatgatattcaccatgtaaatctaatacaatgaaaagtataaatttgaactggaaactaacttagctaaataaatttataaaattaaaaataaaatttaattgggaaatttggaataaaaatatttctataatttatattatgaaaagtaaaatattaaccatgtcgcaatttagtaaatcgaaggttggtatgtgttgtgacacaatctcgtaaattaaaggttggtatgtgtcaccattttgtgagtcacgtttaattgatggtcaacattctccggttaacccccataatgcaaaatcttattttgggcaaatgagagggtaaaatagtcatttaaagaattccgaccggagtttctcctttgattcctcgcccctatactacgcgtcgtgtcagactcgtaactccgtttgactcaattaccccacatttttcattccatcatttcctttttcattgttttcattgtttttggctagttaacatagccaaactatgttcattatgcaactcacgaaacttaatacaaagatgaatctaataactctggttaacgaagtatattctccgttgacaaaaaaaaaaaaaaacacaaacagagaaaagtgaaatctgttgtattaagaactttgtagctcctacaagccttttcatatattgaaaattcacatatttagcaaaagaaaaaaaagaaaattgaaaaatcattcccgttttcatgtactaaaaagttttaacagtaaatatgcatatatttaggaaatgtatagttgccttgataaattggtaagtttgatttaatatgcatttaaatataaaactctttttacatatacttaatcttcaactatcattctaaagtcatacccttttaacactttatttatcctctcttttttttttttggacaactaactttattttatccaatcagcttttaaaatcttatataacatggtttggattggacaagctaagtaataatactataatacgccacgtatatatacgacgtatccttcttctcttcacgtataaaataaaaatagtgtgttattgccataacatacgtgttttctaaaatttaaaagaaagtcatttatcatgaaaaggtatcataatcagaataagagtaatcagtttgaaaactagctagttgactaaaacagaaagaaaaatatctttacttgaaacatgtgttgagaattcacattatataaaaggaaaaaaaataaaacatgacttttgcgtagacaatccatagtccaatcaacaaaacgtgtggacggcaagggttccttctagctctctctatttatatctctcactcgccgatttttactagtaattaaccagaacgttcatctaccaacaaatcctcaactccttcttcttcactaatagtataccacaaaacctcaaaattgtaatttattagattatcgtgatggcaaatatctccagtattcacattctcttcctcgtgttcatcacaagcggcattgctgttatgatgagaagaaactgtaaccttgagcttagacttcttccatcttcttatccatctgattctcatgatatgatggaggagagaattgagtctccaaagactcaacaacaacaacttactattttttataacggaagagtttgtgtttctgatgttactgagcttcaagctaaggctattcttatgcttgctaacagagagagagatgagagaatgaagtctccaactggatgggagccagtttctccaactcttaagtctcaagttaagtgtccaaacactgctctttctatgaagagatctcttcaaagatttcttcaaaagagaaagactagaattcaagctacttctccatatcatatgagcaagggcgaggagctgttcaccggcgtggtgccgatcctggtggagctggacggcgacgtgaacggccacaagttcagcgtgagcggcgagggcgagggcgacgccacctacggcaagctgaccctgaagttcatctgcaccaccggcaagctgccggtgccgtggccgaccctggtgaccaccttcagctacggcgtgcagtgcttcagcaggtacccggaccacatgaagcagcacgacttcttcaagagcgccatgccggagggctacgtgcaggagaggaccatcttcttcaaggacgacggcaactacaagaccagggccgaggtgaagttcgagggcgacaccctggtgaacaggatcgagctgaagggcatcgacttcaaggaggacggcaacatcctgggccacaagctggagtacaactacaacagccacaacgtgtacatcatggccgacaagcagaagaacggcatcaaggtgaacttcaagatcaggcacaacatcgaggacggcagcgtgcagctggccgaccactaccagcagaacaccccgatcggcgacggcccggtgctgctgccggacaaccactacctgagcacccagagcgccctgagcaaggacccgaacgagaagagggaccacatggtgctgctggagttcgtgaccgccgccggcatcacccacggcatggacgagctgtacaagtgatgattaagaaggaaattatttataagaagtaaatgtagaaaacacataaataatttcttgtaaaccatgatatataatatataaagataattttcgtaaagttatgccaaaatcaaaatgactagaaaggttaatgtaaactatcttcttcggtattgacctaagtctagcctgaaatttgagatggcttcttgggttctgacctaacttgggtctcaaactaccttctgatcacacggataaagtataaaccttgaaattataaggtgctatgagaaaaaaaaaatgagagatagaagagtacctttattctcctgggaactcaaggtagagaccctcaattggaaaagagtctcatgactaaattattcccaaaatgttttcggtttcagcttgatgaagaggaagacgtaattgttgaatttgcttatcctttgttgcctccaaaatgttctcgttgtggtaaatggggtcatctaggggatgtgtgcgtgattaaacctcagtctccaatgaaagaaaaagcaatagtagtagaggaaggtgagattgtgtctgttgattctgcaaacataattcagactattccagatactaagactgtcaacaaagttaaagctgtagaagaatataagaattcatagtagttggtagcacagaaattcttagagttgactaaattatagtataaatagttatccattagttctcatagaaacaagaaaacttaaatttatgtcgttgcaaaaccttgctttgtttttgtttagttttcttgttaatcgggtgaattcttgagtccattttaatctccatggtctatagaaattcatgttttctggtcatgcaagacgtttaccgatcgccaaacattttaaaaaatagttgaagaaaatcaagctttaacctaaaatcaacataagaactccaaattttaagcggaagctgctactaccccaccacaacttgatttgcaacactttgccttaatatatataaattctttaagctattttaaatagatctcacgaggttgtatgccttagaaaaaataattatattgttggaagaacgtacaattatcgttgaaactataattatcgttgtacagtacacccatcttttctcgctcattgcggaagctgttgccaattactcgtttatacacatcacgttttgcattattgtttttgtcaactactctatcaattatttatcaaagaaataaaaactttactctataaattatttccgcaaactcaagttatacacgcaaacatcatcatgtccacgtacatttttctactaatgtggaataataacttcacagtaattgaaaccctgatccaaaatcaacaagcacaaaagcgaacgataataatagaacttaaacaacgacacgccaaattctaatttagcacagtctcaacgtgctctaaaaacgagtggtgtaagtgaaaatatctcgttggttacaagcataatgggaccaacaagcatttgcatcactcgaagccatacgtacacttatctccgcattttcccatcggtcttcataagtaatcatacctaacccacctcattatataataccaccctttgtgtcacacaaataaacaccataaactaaacaataaaccatacaaaaaaatatggctaagtttgcttccatcatcacccttatcttcgctgctcttgttctctttgctgctttcgacgcaccggcaatggtggaaatgagaagaaattgtaatcttgaacttagacttcttccttcttcttatccttctgattctcatgatatgatggaagaaagaattgaatctcctaagactcaacaacaacaacttactattttttataatggaagagtttgtgtttctgatgttactgaacttcaagctaaggctattcttatgcttgctaatagagaaagagatgaaagaatgaagtctcctactggatgggaacctgtttctcctactcttaagtctcaagttaagtgtcctaatactgctctttctatgaagagatctcttcaaagatttcttcaaaagagaaagactagaattcaagctacttctccttatcatatggtgagcaagggcgaggaggacaacatggccatcatcaaggagttcatgaggttcaaggtgcacatggagggcagcgtgaacggccacgagttcgagatcgagggcgagggcgagggcaggccgtacgagggcacccagaccgccaagctgaaggtgaccaagggcggcccgctgccgttcgcctgggacatcctgagcccgcagttcatgtacggcagcaaggcctacgtgaagcacccggccgacatcccggactacctgaagctgagcttcccggagggcttcaagtgggagagggtgatgaacttcgaggacggcggcgtggtgaccgtgacccaggacagcagcctgcaggacggcgagttcatctacaaggtgaagctgaggggcaccaacttcccgagcgacggcccggtgatgcagaagaagaccatgggctgggaggccagcagcgagaggatgtacccggaggacggcgccctgaagggcgagatcaagcagaggctgaagctgaaggacggcggccactacgacgccgaggtgaagaccacctacaaggccaagaagccggtgcagctgccgggcgcctacaacgtgaacatcaagctggacatcaccagccacaacgaggactacaccatcgtggagcagtacgagagggccgagggcaggcacagcaccggcggcatggacgagctgtacaagtgatgattaagcatctatcttcatggcattgtccccttgtatccatttcatatctatgtcgtttcgtttatctttgtagccgttttggcaccactgcttaaataaaatgccaatcctatcataactcaataagtacaacgacttcgtactaaattttgtttttcgttaaagggatcattaatcaagtttccatgaaatgatatctccatgtaaatataacagttaacacaacactataccaatctctttaatcatgtatagagtatagactatcattttctatgaatatcttctcaccatttacgttaagctggtagtggtataacactcattcctttacgttatatgatgaaaccaatgtttaaaccgactaatccttaggattgatattagttggga

SEQ ID NO: 2 - protein, 25 aa, Arabidopsis thaliana
MNFTGYSRFLIVFVALVGALVLPSK

SEQ ID NO: 3 - protein, 32 aa, Arabidopsis thaliana
MPPQKENHRTLNKMKTNLFLFLIFSLLLSLSS

SEQ ID NO: 4 - protein, 22 aa, Arabidopsis thaliana
MANISSIHILFLVFITSGIAVM

SEQ ID NO: 5 - protein, 28 aa, Arabidopsis thaliana
MAKFASIITLIFAALVLFAAFDAPAMVE

SEQ ID NO: 6 - DNA, 770 bp, Cauliflower mosaic virus
cgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctct

SEQ ID NO: 7 - DNA, 795 bp, Artificial Sequence
  Synthesized
atggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttc

SEQ ID NO: 8 - DNA, 318 bp, Artificial Sequence
  Synthesized
tgagcgggactctggggttcggatcgatcctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttaca

SEQ ID NO: 9 - DNA, 100 bp, Artificial Sequence
  Synthesized
caaataatgattttattttgactgatagtgacctgttcgttgcaacaaattgatgagcaatgcttttttataatgccaactttgtacaaaaaagcaggct

SEQ ID NO: 10 - DNA, 100 bp, Artificial Sequence
  Synthesized
acccagctttcttgtacaaagttggcattataaaaaataattgctcatcaatttgttgcaacgaacaggtcactatcagtcaaaataaaatcattatttg

SEQ ID NO: 11 - DNA, 25 bp, Artificial Sequence
  Synthesized
acaagtttgtacaaaaaagcaggct

SEQ ID NO: 12 - DNA, 25 bp, Artificial Sequence
  Synthesized
acccagctttcttgtacaaagtggt

SEQ ID NO: 13 - DNA, 21 bp, Artificial Sequence
  Synthesized
tgaccatcttctacaacggca

SEQ ID NO: 14 - DNA, 19 bp, Artificial Sequence
  Synthesized
tgttcgggcacttcacctg

SEQ ID NO: 15 - DNA, 19 bp, Artificial Sequence
  Synthesized
tcctacccatccgactccc

SEQ ID NO: 16 - DNA, 19 bp, Artificial Sequence
  Synthesized
cctctcgtccctctccctg

SEQ ID NO: 17 - DNA, 25 bp, Artificial Sequence
  Synthesized
gacttcttccatcttcttatccatc

SEQ ID NO: 18 - DNA, 26 bp, Artificial Sequence
  Synthesized
ttcattctctcatctctctctctgtt

SEQ ID NO: 19 - DNA, 25 bp, Artificial Sequence
  Synthesized
ccttcttcttatccttctgattctc

SEQ ID NO: 20 - DNA, 28 bp, Artificial Sequence
  Synthesized
cttcattctttcatctctttctctatta

SEQ ID NO: 21 - DNA, 22 bp, Artificial Sequence
  Synthesized
ccgtaaaccttattccattccc

SEQ ID NO: 22 - DNA, 20 bp, Artificial Sequence
  Synthesized
aagaccgctgatgctgtgag

SEQ ID NO: 23 - DNA, 23 bp, Artificial Sequence
  Synthesized
gacccagatcatgtttgagacct

SEQ ID NO: 24 - DNA, 21 bp, Artificial Sequence
  Synthesized
cagtgtggctgacaccatcac

SEQ ID NO: 25 - DNA, 22 bp, Artificial Sequence
  Synthesized
tgagacaagcggtagaaaatgg

SEQ ID NO: 26 - DNA, 18 bp, Artificial Sequence
  Synthesized
cgatgctggggaagacgg

SEQ ID NO: 27 - DNA, 24 bp, Artificial Sequence
  Synthesized
cgctgatagaatgagcaaagaaat

SEQ ID NO: 28 - DNA, 24 bp, Artificial Sequence
  Synthesized
gctgagagaagccaagatagaacc

(19) United States
(12) Patent Application Publication
(10) Pub. No.: US 2025/0215445 A1
(43) Pub. Date: Jul. 3, 2025

(54) PLANT-INDUCED SECRETORY EXPRESSION CASSETTE AND ITS REGULATORY ELEMENTS

(71) Applicant: ZHENGZHOU UNIVERSITY, Zhengzhou City (CN)

(72) Inventors: Fuguang LI, Zhengzhou City (CN); Maozhi REN, Zhengzhou City (CN); HuiJuan MO, Zhengzhou City (CN)

(21) Appl. No.: 18/849,919

(22) PCT Filed: Mar. 29, 2022
(86) PCT No.: PCT/CN2022/083735

§ 371 Date: Sep. 23, 2024

(30) Foreign Application Priority Data
Mar. 21, 2022 (CN) 202210276159.0

(51) Int. Cl. C12N 15/82

(52) U.S. Cl. CPC C12N 15/8271; C12N 15/8237

(57) ABSTRACT

The present disclosure realizes the damage-induced expression of functional genes and verifies that the signal peptide can efficiently mediate the secretion and expression of foreign proteins. Before induction, the expression level of GFP reporter gene in transgenic leaves was very low, which indicated that the promoter of the plant recombinant expression vector containing inducible secretory expression cassette and its regulatory elements was low, and the promoter could start the expression of GFP reporter gene in large quantities during injury induction, which indicated that the promoter responded and induced gene expression was high. In addition, the signal peptide guides the secretion of functional proteins between cells, which helps for overexpressing toxic proteins in plants to improve plant resistance. These characteristics of the inducible secretory expression cassette and its regulatory elements meet the requirements of ideal regulatory elements in plant genetic engineering research and provide valuable materials for plant genetic engineering.

### RELATED APPLICATIONS

The present application is a U.S. National Phase of International Application Number PCT/CN2022/083735 filed on Mar. 29, 2022, which claims priority to Chinese Application Number 202210276159.0 filed on Mar. 21, 2022.

### INCORPORATION BY REFERENCE

The sequence listing provided in the file entitled GNP22006NSequence-2022032404_rev2.txt which is an ASCII text file that was created on Sep. 23, 2024, and which comprises 20,195 bytes, is hereby incorporated by reference in its entirety.

### TECHNICAL FIELD

The present disclosure belongs to the field of genetic engineering, and particularly relates to a plant induced secretory expression cassette and a regulation element thereof.

### BACKGROUND

Jasmonic acid (JA) and its derivatives are a class of lipid hormones containing oxygen, which are ubiquitous in the plant kingdom and play an important role in plant development and response to abiotic stresses and pathogens (Yang Z, Huang Y, Yang J, Yao S, Zhao K, Wang D, Qin Q, Bian Z, Li Y, Lan Y, Zhou T, Wang H, Liu C, Wang W, Qi Y, Xu Z, Li Y. Jasmonate Signaling Enhances RNA Silencing and Antiviral Defense in Rice. Cell Host Microbe. 2020 Jul. 8; 28(1):89-103.e8. doi: 10.1016/j.chom.2020.05.001. Epub 2020 Jun. 5. PMID: 32504578.). After the host plant is subjected to mechanical damage or insect feeding, the JA content in the plant is induced to be rapidly synthesized in large quantities, and the expression of JA synthesis pathway genes including LOX2, VSP2, LOX3, VSP1, JAR1, AOS and other genes will be rapidly up-regulated, which will start the downstream defense mechanism (Ye M, Glauser G, Lou Y, Erb M, Hu L. Molecular Dissection of Early Defense Signaling Underlying Volatile-Mediated Defense Regulation and Herbivore Resistance in Rice. Plant Cell. 2019 March; 31(3):687-698. doi: 10.1105/tpc.18.00569. Epub 2019 Feb. 13. PMID: 30760558; PMCID: PMC6482627.). Therefore, using the promoter of the gene efficiently induced by methyl jasmonate to drive the functional genes in plants for genetic improvement of crops can make transgenic plants drive the efficient expression of functional genes in plants after sensing the damage signal, thus giving transgenic plants good defense ability.

In genetic engineering, the regulatory elements of plant diseases and insect pests injury-induced gene expression include promoter, 5′UTR, 3′UTR and terminator, as well as signal peptide sequences with high secretion and expression, which are very important for inducing high expression of target genes (Lu Y, Rijzaani H, Karcher D, Ruf S, Bock R. Efficient metabolic pathway engineering in transgenic tobacco and tomato plastids with synthetic multigene operons. Proc Natl Acad Sci USA. 2013 Feb. 19; 110(8):E623-32. doi: 10.1073/pnas.1216898110. Epub 2013 Feb. 4. PMID: 23382222; PMCID: PMC3581966.).

Promoter is an important genomic regulatory element that directly affects the gene expression level. It is usually located in the upstream of the 5′-terminal of the gene, which can start transcription correctly and effectively and play a key role in the process of DNA transcription into RNA. In plant genetic engineering, promoters are divided into three types according to different gene expression modes: constitutive, inducible and tissue-specific promoters. Inducible promoter means that the expression of the target gene is greatly increased under the conditions of hormone and stress (Li Tiantian, Li Lili, Chen Lihong, Gao Lifen. Cloning and functional identification of inducible promoters of methyl jasmonate and Xanthomonas oryzae PV. oryzae in rice [J]. Molecular Plant Breeding, 2018,16 (03):689-695. DOI:10.13271/j.mpb.016.000689.). Therefore, it is of great significance to develop the transformation system of this kind of inducible promoter for plant transgenic breeding, which can make foreign genes express under specific induction conditions, which can not only improve the expression level of foreign genes in transgenic plants, but also avoid the waste of resources caused by unnecessary metabolism.

In addition to the promoter, 5′UTR, 3′UTR and other elements and signal peptide will also affect the protein expression (patent: CN103114102B). For the protein expression system, several key features are needed. For example, the system should have negligible harmful effects on the viability and integrity of the host cells. Therefore, when expressing the toxic protein with insecticidal characteristics, it should be considered to avoid damage to plant cells, and it should be necessary to add signal peptide sequence to the amino acid sequence of the recombinant target protein to realize secretion. Under this background, the inventors have studied secretory proteins and genes in plants, such as Arabidopsis PR1, PR3, PR5, PDF1.2, etc. The amino acid sequences of these genes have the function of signal peptides, which can guide the secretion and release of proteins from host cells. The target protein can be any target protein that technicians can understand, and the promoter characteristics can be improved by combining different cis-regulatory elements. With the combination of elements, the target protein can be formed to ensure highly specialized, controllable and satisfying damage-induced secretory expression in different plant species.

### SUMMARY

The technical problem to be solved by the present disclosure is how to make functional genes respond quickly and secrete and express functional proteins after plants are induced by insect damage and/or disease damage and/or mechanical damage.

In order to solve the above technical problems, the present disclosure provides a DNA molecule (multi-gene expression cassette), which includes four expression cassettes, and each expression cassette respectively includes a promoter, a 5′UTR connected with the promoter, a signal peptide coding gene connected with the 5′UTR, a functional gene connected with the signal peptide coding gene, a 3′UTR connected with the functional gene and a terminator connected with the 3′UTR;

The promoter, 5′UTR, 3′UTR and terminator are all derived from jasmonic acid-induced expression genes; the signal peptide is derived from secretory protein;

The nucleotide sequences (such as coding sequences) of functional genes in each expression cassette are different, and the nucleotide sequences of promoters, 5′UTR, 3′UTR, signal peptide coding genes and terminators in each expression cassette are different or the same.

The jasmonic acid-induced expression genes in the present disclosure include but are not limited to: AtLOX2, AtVSP2, AtLOX3, AtVSP1, AtJAR1, AtAOS, AtMYC2, etc.

In the present disclosure, the secretory proteins include but are not limited to: AtPR1, AtPR3, atr5, AtPDF1.2, AtPIP1, etc.

In the present disclosure, the amino acid sequences of protein encoded by the functional genes may be the same or different.

Further, in the above DNA molecule, the four expression cassettes are expression cassette A, expression cassette B, expression cassette C and expression cassette D;

The promoter of the expression cassette A is p-AtLOX2, and the nucleotide sequence of the p-AtLOX2 is the 1st-896th positions of SEQ ID No. 1; the 5′UTR of the expression cassette A is AtLOX2-5′UTR, and the nucleotide sequence of the AtLOX2-5′UTR is the 897th-1088th positions of SEQ ID No. 1; the 3′UTR of the expression cassette A is AtLOX2-3′UTR, and the nucleotide sequence of the AtLOX2-3′UTR is the 1548th-1672nd positions of SEQ ID No. 1; the terminator of the expression cassette a is t-AtLOX2, and the nucleotide sequence of the t-AtLOX2 is 1673rd-1872nd positions of SEQ ID No. 1;

the promoter of the expression cassette B is p-AtVSP2, and the nucleotide sequence of the p-AtVSP2 is the 1873rd-2964th positions of SEQ ID No. 1; the 5′UTR of the expression cassette B is AtVSP2-5′UTR, and the nucleotide sequence of the AtVSP2-5′UTR is the 2965th-3344th positions of SEQ ID No. 1; the 3′UTR of the expression cassette B is AtVSP2-3′UTR, and the nucleotide sequence of the AtVSP2-3′UTR is the 3829th-4078th positions of SEQ ID No. 1; the terminator of the expression cassette B is t-AtVSP2, and the nucleotide sequence of the t-AtVSP2 is the 4079th-4278th positions of SEQ ID No. 1;

the promoter of the expression cassette C is p-AtLOX3, and the nucleotide sequence of the p-AtLOX3 is 4279th-5278th positions of SEQ ID No. 1; the 5′UTR of the expression cassette C is AtLOX3-5′UTR, and the nucleotide sequence of the AtLOX3-5′UTR is the 5279th-5467th positions of SEQ ID No. 1; the 3′UTR of the expression cassette C is AtLOX3-3′UTR, and the nucleotide sequence of the AtLOX3-3′UTR is the 5892nd-6747th positions of SEQ ID No. 1; the terminator of the expression cassette C is t-AtLOX3, and the nucleotide sequence of the t-AtLOX3 is 6748th-6947th positions of SEQ ID No. 1;

the promoter of the expression cassette D is p-AtVSP1, and the nucleotide sequence of the p-AtVSP1 is the 6948th-7947th positions of SEQ ID No. 1; the 5′UTR of the expression cassette D is AtVSP1-5′UTR, and the nucleotide sequence of the AtVSP1-5′UTR is the 7948th-8259th positions of SEQ ID No. 1; the 3′UTR of the expression cassette D is AtVSP1-3′UTR, and the nucleotide sequence of the AtVSP1-3′UTR is the 9409th-9607th positions of SEQ ID No. 1; the terminator of the expression cassette D is t-AtVSP1, and the nucleotide sequence of the t-AtVSP1 is the 9608th-9807th positions of SEQ ID No. 1

Further, in the above DNA molecule, the signal peptide is selected from sp-AtPR1, sp-AtPR3, sp-AtPR5 and sp-AtPDF1.2; the amino acid sequence of sp-AtPR1 is SEQ ID No.2, the amino acid sequence of sp-AtPR3 is SEQ ID No.3, the amino acid sequence of sp-AtPR5 is SEQ ID No.4, and the amino acid sequence of sp-AtPDF1.2 is SEQ ID No.5.

Further, the signal peptide coding gene is the coding gene of sp-AtPR1, sp-AtPR3, sp-AtPR5 or sp-AtPDF1.2, and the coding gene of sp-AtPRI is a DNA molecule with a nucleotide sequence of 1089th-1163rd positions of SEQ ID No. 1; the coding gene of sp-AtPR3 is a DNA molecule with a nucleotide sequence of 3345th-3440th positions of SEQ ID No. 1; the coding gene of sp-AtPR5 is a DNA molecule with a nucleotide sequence of 5468th-5533rd position of SEQ ID No. 1; the encoding gene of sp-AtPDF1.2 is a DNA molecule with the nucleotide sequence of 8260th-8343rd positions of SEQ ID No. 1

In order to solve the above technical problems, in a second aspect, the present disclosure provides a recombinant vector containing the above DNA molecule.

In order to solve the above technical problems, in a third aspect, the present disclosure provides a recombinant microorganism containing the above DNA molecule or containing the above recombinant vector;

In order to solve the above technical problems, in a fourth aspect, the present disclosure provides a transgenic plant cell line or/and a transgenic plant tissue or/and a transgenic plant organ containing the above DNA molecule or the above recombinant vector. The recombinant microorganism can be yeast, bacteria, algae and fungi.

The above plant tissues can be derived from roots, stems, leaves, flowers, fruits, seeds, pollen, embryos and anthers.

The organs of transgenic plants can be roots, stems, leaves, flowers, fruits and seeds of transgenic plants.

Among the above related biological materials, the transgenic plant cell lines, transgenic plant tissues and transgenic plant organs may or may not include propagation materials.

In order to solve the above technical problems, in a fifth aspect, the present disclosure provides a use which is any one of a1) to a3):

A1) use of the DNA molecule of claim 1 in damage-induced expression of plant functional genes;

A2) use of the DNA molecule according to claim 1 in improving the expression of plant functional genes; and

A3) use of the DNA molecule according to claim 1 in plant stress resistance. Further, in the above-mentioned use, the use may specifically be:

C1) a method for making plants express functional genes under injury induction, which includes introducing the DNA molecule into recipient plant to obtain a transgenic plant, and the transgenic plant expressing the functional genes under injury induction.

C2) a method for producing a transgenic plant, which includes introducing the above DNA molecule into a recipient plant to obtain a transgenic plant, and the transgenic plant expressing the functional genes under injury induction.

C3) a method for up-regulating the expression of functional genes in a plant under injury induction, which includes introducing the above DNA molecule into a recipient plant to up-regulate the expression of functional genes in the recipient plant under injury induction.

Further, the improvement of plant stress resistance can be to improve the damage-induced expression of plant functional genes.

In the present disclosure, the biomaterial can be a recombinant vector, a recombinant microorganism, a transgenic plant cell line, a transgenic plant tissue or a transgenic plant organ containing the above DNA molecule.

In order to solve the above technical problems, in a sixth aspect, the present disclosure provides a method for improving the stress resistance of plants, which includes expressing a functional gene in a recipient plant with the above expression cassette to improve the stress resistance of the recipient plant.

Further, in the above method, the functional gene is a damage-induced gene.

In the above method, the damage-induced gene includes, but is not limited to, any functional gene such as GhJAZ8.

Illustratively, the damage-inducing gene in the above method includes but is not limited to any one of G1)-G4):

G1), optimizing the nucleotide sequence of GhJAZ8 according to the codon preference of corn GhJAZ8-Maize (the nucleotide sequence is 1164th-1520th positions of SEQ ID No. 1);

G2) optimizing the nucleotide sequence of GhJAZ8 according to the codon preference of wheat GhJAZ8-Wheat (the nucleotide sequence is the 3441st-3797th positions of SEQ ID No. 1);

G3) optimizing the nucleotide sequence of GhJAZ8 according to the codon preference of soybean GhJAZ8-soybean (the nucleotide sequence is the 5534th-5890th positions of SEQ ID No. 1);

G4), optimizing the nucleotide sequence of GhJAZ8 according to the codon preference of rice GhJAZ8-Rice (the nucleotide sequence is the 8344th-8700th positions of SEQ ID No. 1).

In the above method, the recipient plant includes but is not limited to corn, sorghum, wheat, sunflower, tomato, pepper, potato, cotton, rice, soybean, beet, sugarcane, tobacco and barley.

### BRIEF DESCRIPTION OF DRAWINGS

FIG. 1 is an element diagram of functional genes int the regulated plant.

FIG. 2 is a schematic diagram of a skeleton vector containing an inducible secretory expression cassette and its regulatory elements.

FIG. 3 is a schematic diagram of a plant binary expression vector containing an inducible secretory expression cassette and its regulatory elements.

FIG. 4 shows the PCR identification of transgenic plants such as rice, corn, cotton and tobacco.

FIGS. 5A-5D shows the experimental results of qRT-PCR to determine the expression of all four expression cassettes.

FIG. 6 shows the treatment of puncture injury of leaves of a transgenic plant GMO rice and non-transgenic rice.

FIG. 7 shows the puncture injury treatment of leaves of a transgenic plant GMO corn and non-transgenic plant B73 corn.

FIG. 8 shows the puncture injury treatment of transgenic plant GMO cotton.

FIG. 9 shows the puncture injury treatment of transgenic plant GMO tobacco.

FIG. 10 is the secretory evidence of inducible secretory expression cassette and its regulatory elements.

### DESCRIPTION OF EMBODIMENTS

The present disclosure will be further described in detail with reference to specific embodiments, and the examples are given only to illustrate the present disclosure, but not to limit the scope of the present disclosure. The examples provided below can be used as a guide for further improvement by those skilled in the art, and are not intended to limit the present disclosure in any way.

Unless otherwise specified, the experimental methods in the following examples are all conventional methods, which are carried out according to the techniques or conditions described in the literature in this field or according to the product specifications. The materials and reagents used in the following examples can be commercially available unless otherwise specified.

### Example 1. Acquisition of Damage-Inducing Promoter and Terminator Elements

The expression genes of Arabidopsis thaliana induced by jasmonic acid were obtained by Genevestigator software, and the false positive genes in the data were removed by EST mode in UniGene. Four genes, AtLOX2, AtVSP2, AtLOX3 and AtVSP1, were randomly selected. The promoter region sequences of these genes were obtained from TAIR (The Arabidopsis Information Resource, http://www.arabidopsis.org/) website. An online promoter prediction tool, PlantCARE (plant cis-acting regulatory elements, http://www.bioinformatics.psb.ugent.be/webtools/plantcare/html), was used to make analysis, showing that the promoter sequences of these four genes contain the basic promoter structure. Then, the 5′UTR, 3′UTR and terminator sequences of the four genes were obtained from TAIR website. The DNA fragments of Arabidopsis thaliana with promoter function in response to jasmonic acid-induced AtLOX2 (Gene ID AT3G45140), AtVSP2 (Gene ID AT5G24770), AtLOX3 (Gene ID AT1G17420) and AtVSP1 (Gene ID AT5G24780) were named as promoters p-AtLOX2, p-AtVSP2, p-AtLOX3 and p-AtVSP1, and the nucleotide sequences thereof are 1st-896th positions of SEQ ID No.1, 1873rd-2964th positions of SEQ ID No.1, 4279th-5278th positions of SEQ ID No.1 and 6948th-7947th positions of SEQ ID No.1. The sequences of 5′UTR cis-regulatory elements of AtLOX2, AtVSP2, AtLOX3 and AtVSP1 genes are 897th-1088th positions of SEQ ID No.1, 2965th-3344th positions of SEQ ID No.1, 5279th-5467th positions of SEQ ID No.1 and 7948th-8259th positions of SEQ ID No.1 respectively. The sequences of 3′UTR cis-regulatory elements of AtLOX2, AtVSP2, AtLOX3 and AtVSP1 genes are the 1548th-1672nd, 3829th-4078th, 5892nd-6747th and 9409th-9667th positions of SEQ ID No. 1, respectively. The DNA fragments of AtLOX2, AtVSP2, AtLOX3 and AtVSP1 genes with terminator function are named as terminators t-AtLOX2, t-AtVSP2, t-AtLOX3 and t-AtVSP1 respectively, and their nucleotide sequences are 1673rd-1872nd positions of SEQ ID No. 1, 4079th-4278th of positions of SEQ ID No. 1, 6748th-6947th positions of SEQ ID No. 1 respectively.

### Example 2: Acquisition of Secretory Signal Peptide Element

The gene sequence of secretory protein in Arabidopsis thaliana was searched through literature and TAIR database, the signal peptide of the protein was predicted by using SignalP5.0 online prediction service (http://www.cbs.dtu.dk/services/SignalP/), and the protein with the signal peptide was further screened according to the prediction result. Finally, four protein genes AtPR1 (Gene ID AT2G14610), AtPR3 (Gene ID AT3G12500), AtPR5 (Gene ID AT1G75040) and ATPF1.2 (Gene ID AT5G4420) with high protein expression and signal peptide were screened. The signal peptides were named sp-AtPR1, sp-AtPR3, sp-AtPR5 and sp-AtPDF1.2 respectively. The amino acid sequences of the signal peptides were SEQ ID No.2, SEQ ID No.3, SEQ ID No.4 and SEQ ID No.5 respectively, and the nucleotide sequences encoding the signal peptides were 1089-1163, 3345-3440, 5468-5533 or 8260-8343 positions of SEQ ID No. 1 respectively.

### Example 3: Construction of Plant Recombinant Expression Vector for Inducing Secretory Expression Cassette and Its Regulatory Elements

Four expression cassettes of cotton GhJAZ8 (the gene ID in cotton database (https://cottonfgd.org/analyze/) is Gh_A05G1241, Sun H, Chen L, Li J, Hu M, Ullah A, He X, Yang X, Zhang X. The JASMONATE ZIM-Domain Gene Family Mediates JA Signaling and Stress Response in Cotton. Plant Cell Physiol. 2017 Dec. 1; 58(12):2139-2154. doi: 10.1093/pcp/pcx148. PMID: 29036515.) plant recombinant expression vector were constructed. The elements of each expression cassette are a promoter, a 5′UTR connected to the promoter, a signal peptide coding gene connected to the 5′UTR, a functional gene connected with the signal peptide coding gene, a 3′UTR connected with the functional gene and a terminator connected with the 3′UTR.

In addition, each GhJAZ8 gene sequence was optimized by the codon preference of different crops, and the nucleotide sequence of GhJAZ8-Maize was optimized according to the codon preference of maize (the nucleotide sequence is the 1164th-1520th position of SEQ ID No. 1).

The nucleotide sequence of GhJAZ8 was optimized according to the codon preference of corn GhJAZ8-Maize (the nucleotide sequence is 1164th-1520th positions of SEQ ID No. 1); the nucleotide sequence of GhJAZ8 was optimized according to the codon preference of wheat GhJAZ8-Wheat (the nucleotide sequence is the 3441st-3797th positions of SEQ ID No. 1); the nucleotide sequence of GhJAZ8 was optimized according to the codon preference of soybean GhJAZ8-soybean (the nucleotide sequence is the 5534th-5890th positions of SEQ ID No. 1); the nucleotide sequence of GhJAZ8 was optimized according to the codon preference of rice GhJAZ8-Rice (the nucleotide sequence is the 8344th-8700th positions of SEQ ID No. 1).

Expression cassette A: p-atlox2, AtLOX2-5′UTR, sp-AtPR1, GhJAZ8-Maize gene, HA tag, AtLOX2-3′UTR, T-atlox2;

Expression cassette B: p-atvsp2, AtVSP2-5′UTR, sp-AtPR3, GhJAZ8-Wheat gene, CMYC tag, AtVSP2-3′UTR, t-atvsp2;

Expression cassette C: p-atlox3, AtLOX3-5′UTR, sp-AtPR5, GhJAZ8-soybean gene, GFP tag, AtLOX3-3′UTR, t-atlox3;

Expression cassette D: P-ATV SP1, AtVSP1-5′UTR, sp-PDF1.2, GhJAZ8-Rice gene, mCherry tag, AtVSP1-3′UTR, t-AtVSP1.

Expression cassette A: p-atlox2 (the nucleotide sequence is positions 1-896 of SEQ ID No. 1), AtLOX2-5′UTR (tge nucleotide sequence is positions 897-1088 of SEQ ID No. 1, sp-AtPR1 (the nucleotide sequence is positions 1089-1163 of SEQ ID No. 1), GhJAZ8-Maize gene (the nucleotide sequence is positions 1164-1520 of SEQ ID No. 1), HA tag (the nucleotide sequence is positions 1521-1547 of SEQ ID No. 1), AtLOX2-3′UTR (the nucleotide sequence is positions 1548-1672 of SEQ ID No. 1) and t-AtLOX2 (the nucleotide sequence is positions 1673-1872 of SEQ ID No. 1)

Expression cassette B: p-atvsp2 (the nucleotide sequence is positions 1873-2964 of SEQ ID No. 1), AtVSP2-5′UTR (the nucleotide sequence is positions 2965-3344 of SEQ ID No. 1), sp-AtPR3 (the nucleotide sequence is positions 3345-3440 of SEQ ID No. 1), GhJAZ8-Wheat gene (the nucleotide sequence is positions 3441-3797 of SEQ ID No. 1), CMYC tag (the nucleotide sequence is positions 3798-3828 of SEQ ID No. 1), AtVSP2-3′UTR (the nucleotide sequence is positions 3829-4078 of SEQ ID No. 1) and t-AtVSP2 (the nucleotide sequence is positions 4079-4278 of SEQ ID No. 1).

Expression cassette C: p-atlox3 (the nucleotide sequence is 4279-5278 of SEQ ID No. 1), AtLOX3-5′UTR (the nucleotide sequence is positions 5279-5467 of SEQ ID No. 1), sp-AtPR5 (the nucleotide sequence is positions 5468-5533 of SEQ ID No. 1), GhJAZ8-soybean gene (the nucleotide sequence is positions 5534-5890 of SEQ ID No. 1), GFP tag (the nucleotide sequence is positions 5891-6604 of SEQ ID No. 1), AtLOX3-3′UTR (the nucleotide sequence is positions 5892-6747 of SEQ ID No. 1), and t-AtLOX3 (the nucleotide sequence is positions 6748-6947 of SEQ ID No. 1).

Expression cassette D: P-ATV SP1 (the nucleotide sequence is positions 6948-7947 of SEQ ID No. 1), AtVSP1-5′UTR (the nucleotide sequence is positions 7948-8259 of SEQ ID No. 1), sp-PDF1.2 (the nucleotide sequence is positions 8260-8343 of SEQ ID No. 1), GhJAZ8-Rice gene (the nucleotide sequence is positions 8344-8700 of SEQ ID No. 1), mCherry tag (the nucleotide sequence is positions 8701-9408 of SEQ ID No. 1), AtVSP1-3′UTR (the nucleotide sequence is positions 9409-9607 of SEQ ID No. 1) and t-AtVSP1 (the nucleotide sequence is positions 9608-9807 of SEQ ID No. 1).

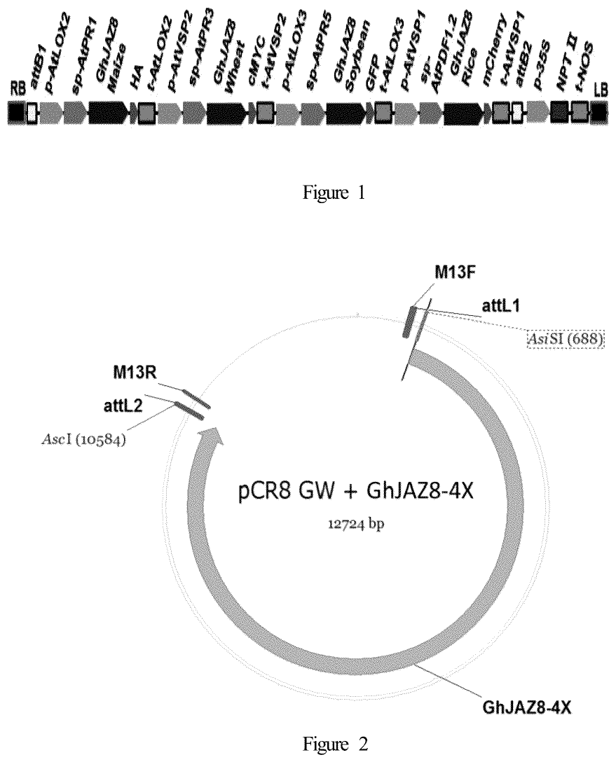

HA, CMYC, GFP and mcherry were used as protein tags of the four expression cassettes, and the four expression cassettes were sorted in turn. See FIG. 1 for their structures.

In FIG. 1, the prefix p denotes a promoter (containing a 5′UTR sequence); the prefix sp represents a signal peptide; the prefix t stands for terminator (containing 3′UTR sequence); HA (the nucleotide sequence is positions 1521-1547 of SEQ ID No. 1), cMYC (the nucleotide sequence is positions 3798-3828 of SEQ ID No. 1), GFP (the nucleotide sequence is positions 5891-6604 of SEQ ID No. 1) and mCherry (the nucleotide sequence is positions 8701-9408 of SEQ ID No. 1). P-35S is a constitutive expression promoter (SEQ ID No.6), NPTII is a resistance marker (SEQ ID No.7) and t-NOS is a terminator (SEQ ID No.8).

Then, the whole element regulating the functional genes in plants was synthesized (Shanghai Sangon). The synthesized whole sequence was named GhJAZ8-4X, and its nucleotide sequence was SEQ ID No. 1 GhJAZ8-4X was constructed into M35S-8GWN vector, and the obtained vector was named pCR8GW+GhJAZ8-4X (FIG. 2).

FIG. 2 is a schematic diagram of a skeleton vector containing an inducible secretory expression cassette and its regulatory elements. The skeleton vector is M35S-8GWN (pCR8GW, Invitrogen, article number K250020), and GhJAZ8-4X refers to four multi-gene expression cassettes of GhJAZ8. attL1 (SEQ ID No.9) and attL2 (SEQ ID No. 10) will be used for LR reaction when constructing recombinant plant binary expression vectors.

The multigene expression cassette was connected to the target expression vector pMDC100 (BioVector, article number: CD3-746) by GatewayLR reaction, and the obtained recombinant vector was named PMDC100-LR1-GhJAZ8-4X (FIG. 3).

FIG. 3 is a schematic diagram of a plant binary expression vector containing an inducible secretory expression cassette and its regulatory elements. The skeleton vector is pMDC100, and attB1 (SEQ ID No. 11) and attB2 (SEQ ID No. 12) are the recombinant sequences after the skeleton vector and the expression vector LR. CaMV35Spromoter is a constitutive expression promoter (SEQ ID No.6), NPTII is a resistance marker (SEQ ID No.7), and t-NOS is a terminator (SEQ 15IDNo.8).

### Example 4: Acquisition and Identification of Transgenic Plants

The recombinant expression vector plasmid pMDC100-LR1-GhJAZ8-4X, which contains inducible secretory expression cassette and its regulatory elements, was introduced into Agrobacterium LBA4404 strain and subjected to genetic transformation of rice, maize, cotton and tobacco. The above plant expression vectors were introduced into recipient plants by Agrobacterium-mediated hypocotyl transformation of rice, corn, cotton and tobacco.

The recipient variety of rice is Japanese Haruki, and the transformation method is referred to in the literature: Zhao W, Zheng S, Ling H Q. An efficient regeneration system and Agrobacterium-mediated transformation of Chinese upland rice cultivar Handao297. Plant Cell Tissue & Organ Culture. 2011, 106(3):475.

The recipient variety of maize is B73, and the transformation method refers to the literature: Lee H, Zhang Z J. Agrobacterium-mediated transformation of maize (Zea mays) immature embryos. Methods Mol Biol. 2014; 1099:273-280.

The recipient variety of cotton is ZM24, and the transformation method refers to the literature: Yang Z, Ge X, Yang Z, Qin W, Sun G, Wang Z, Li Z, Liu J, Wu J, Wang Y, Lu L, Wang P, Mo H, Zhang X, Li F. Extensive intraspecific gene order and gene structural variations in upland cotton cultivars. Nat Commun. 2019 Jul. 5; 10(1):2989. doi: 10.1038/s41467-019-10820-x. PMID: 31278252; PMCID: PMC6611876.

The recipient variety of tobacco is Benji tobacco, and the transformation method refers to the literature: Sunilkumar G, Vijayachandra K, Veluthambi K. Preincubation of cut tobacco leaf explants promotes Agrobacterium-mediated transformation by increasing vir gene induction. Plant Science, 1999. 141(1):51-58.

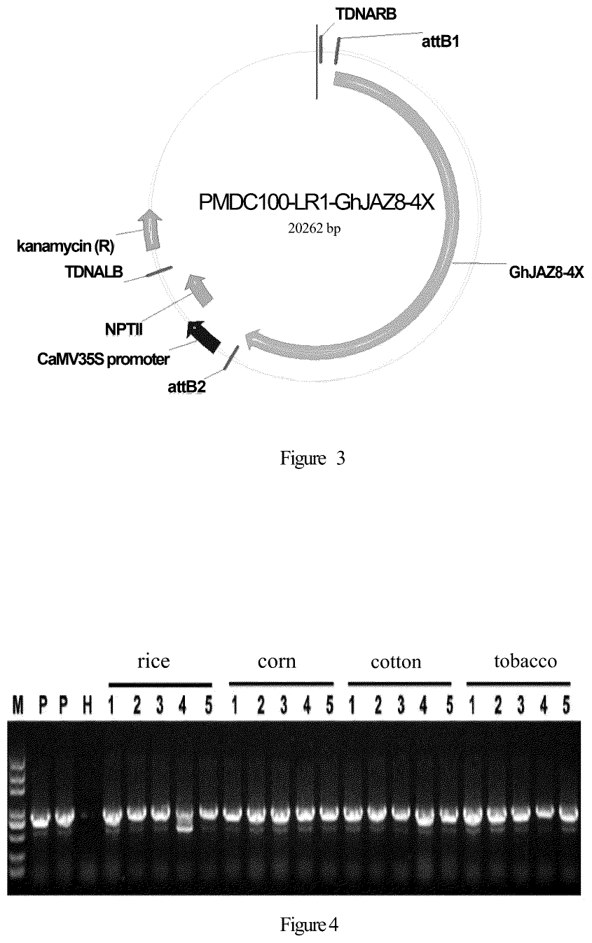

There were 20 recipient plants in each group. When rice, corn, cotton and tobacco plants were transplanted and grew to a certain size, 0.5 g leaves was taken to extract genomic DNA of rice, corn, cotton and tobacco, and amplify the over-expressed transgenic rice, corn, cotton and tobacco with cross-vector primers (JAZ7: ggtttacccgccaatatatcc, JAZ7: tcaattcgaacatggctataac, PCR product size is 967 bp). The electrophoresis result is shown in FIG. 4, in which M is DNAmarker, and from top to bottom is 5000, 3000, 2000, 1000, 750, 500, 250 and 100 bp. The size of the PCR product is 967 bp. 1-5 are the numbers of transgenic lines. P is plasmid positive control. His water negative control.

FIG. 4 shows that transgenic rice, maize, cotton and tobacco plants can amplify a band consistent with the positive control, indicating that the T-DNA segment of plant expression vector has been integrated into the genomes of transgenic rice, maize, cotton and tobacco, and 5 positive plants were obtained.

### Example 5. Identification of Damage-Induced Characteristics of Inducible Secretory Expression Cassettes and Their Regulatory Elements

5.1. The Expression of Four Expression Cassettes was Determined by qRT-PCR.

RNA was extracted from the leaves of GMO rice, corn, cotton and tobacco, and the expression of four expression cassettes was determined by qRT-PCR.

The relative expression levels of the target gene GhJAZ8 in expression cassettees A, B, C and D in GMO cotton were determined with GhTub1 as the reference gene, qBox A-F and qBox A-R, qBox B-Fand qBox, B-R, qBox C-F and qBox C-R, Box D-F and qBox D-R as the primers respectively.

The relative expression levels of the target gene GhJAZ8 in expression cassettees A, B, C and D in GMO rice were determined with OsActin as the reference gene, qBox A-F and qBox A-R, qBox B-Fand qBox, B-R, qBox C-F and qBox C-R, Box D-F and qBox D-R as the primers respectively.

The relative expression levels of the target gene GhJAZ8 in expression cassettees A, B, C and D in GMO corn were determined with ZmActin as the reference gene, qBox A-F and qBox A-R, qBox B-Fand qBox, B-R, qBox C-F and qBox C-R, Box D-F and qBox D-R as the primers respectively.

The relative expression levels of the target gene GhJAZ8 in expression cassettees A, B, C and D in GMO tobacco were determined with NbActin as the reference gene, qBox A-Fand qBox A-R, qBox B-F and qBox, B-R, qBox C-F and qBox C-R, Box D-F and qBox D-R as the primers respectively.

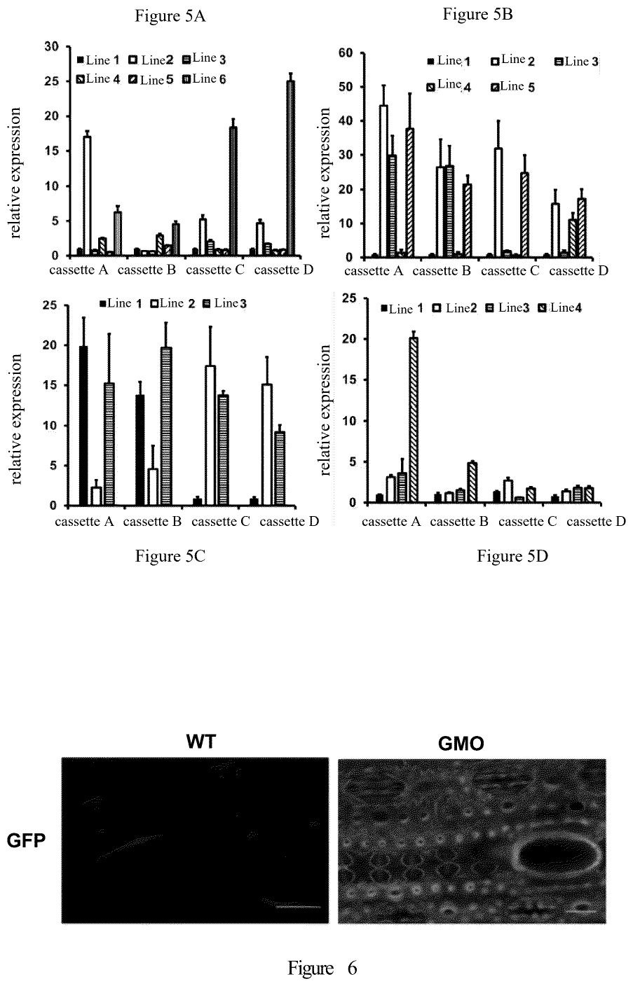

The primer sequence of qRT-PCR is shown in Table 1, and the result of qRT-PCR is shown in FIGS. 5A-5D. In FIGS. 5A-5D, FIG. 5A is the relative expression level of the target gene in four expression cassettees in transgenic rice, FIG. 5B is the relative expression level of the target gene in four expression cassettees in transgenic cotton, FIG. 5C is the relative expression level of the target gene in four expression cassettees in transgenic corn, and FIG. 5D is the relative expression level of the target gene in four expression cassettees in transgenic tobacco.

FIGS. 5A-5D shows that all four expression cassettes of transgenic rice, maize, cotton and tobacco plants can be expressed, indicating that the functional protein has been successfully expressed in the genomes of transgenic rice, maize, cotton and tobacco.

TABLE 1
primer sequences used to determine the expression of four expression
cassettes by 1qRT-PCR method.

| Gene | | | |
|---|---|---|---|
| Name | Primer Name | Primer sequences with ID numbers (5′-3′) | Gene ID |
| Cassette | qBox A-F | TGACCATCTTCTACAACGGCA (SEQ ID No. 13) | / |
| A | qBox A-R | TGTTCGGGCACTTCACCTG (SEQ ID No. 14) | |
| Cassette | qBox B-F | TCCTACCCATCCGACTCCC (SEQ ID No. 15) | / |
| B | qBox B-R | CCTCTCGTCCCTCTCCCTG (SEQ ID No. 16) | |
| Cassette | qBox C-F | GACTTCTTCCATCTTCTTATCCATC (SEQ ID No. 17) | / |
| C | qBox C-R | TTCATTCTCTCATCTCTCTCTCTGTT (SEQ ID No. 18) | |
| cassette | qBox D-F | CCTTCTTCTTATCCTTCTGATTCTC (SEQ ID No. 19) | / |
| D | qBox D-R | CTTCATTCTTTCATCTCTTTCTCTATTA (SEQ ID No. 20) | |
| GhTub1 | qTub1-F | CCGTAAACCTTATTCCATTCCC (SEQ ID No. 21) | AF484959.1 |
| | qTub1-R | AAGACCGCTGATGCTGTGAG (SEQ ID No. 22) | |
| OsActin | qOsActin-F | GACCCAGATCATGTTTGAGACCT (SEQ ID No. 23) | AY212324.1 |
| | qOsActin-R | CAGTGTGGCTGACACCATCAC (SEQ ID No. 24) | |
| ZmActin | qZmActin-F | TGAGACAAGCGGTAGAAAATGG (SEQ ID No. 25) | XM_020548264.3 |
| | qZmActin-R | CGATGCTGGGGAAGACGG (SEQ ID No. 26) | |
| NbActin | qNbActin-F | CGCTGATAGAATGAGCAAAGAAAT (SEQ ID No. 27) | JQ256516.1 |
| | qNbActin-R | GCTGAGAGAAGCCAAGATAGAACC (SEQ ID No. 28) | |

### 5.2 Damage-Induced Expression of Four Expression Cassettes

The leaves of GMO rice, corn, cotton and tobacco were treated with puncture injury respectively. Compared with the wild-type rice leaves, the strong fluorescent signal of GFP can be observed 20 minutes after GMO rice leaves were punctured, but the strong fluorescent signal of GFP was not observed in the wild-type rice leaves punctured (FIG. 6).

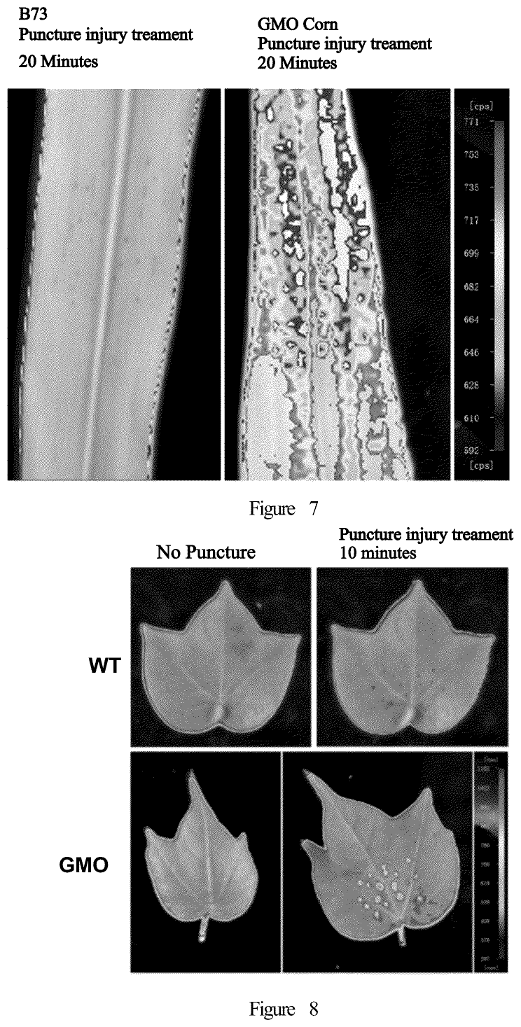

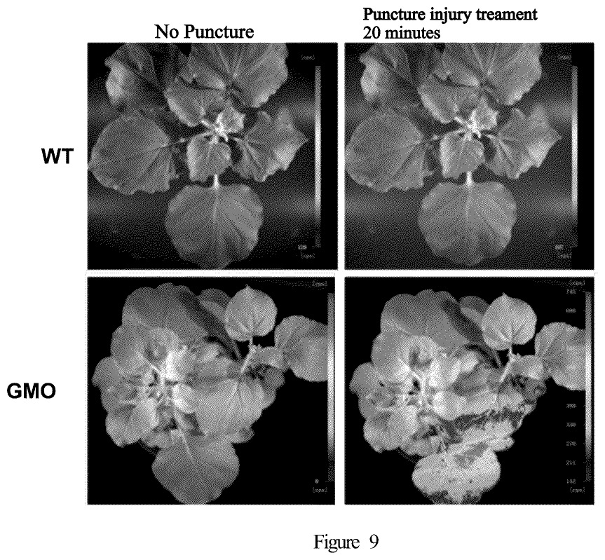

Compared with wild-type maize leaves, the strong fluorescent signal of GFP can be observed 20 minutes after GMO maize leaves were punctured, but the strong fluorescent signal of GFP was not observed in wild-type maize leaves punctured (FIG. 7). Experiments on adjacent leaves from the same GMO cotton plant found that the strong fluorescent signal of GFP was not observed in the leaves of GMO cotton without punctures, in contrast, the strong fluorescent signal of GFP was observed 10 minutes after the punctures were punched in the leaves of GMO cotton (FIG. 8). The puncture injury experiment on two adjacent leaves of the whole GMO tobacco plant showed that the strong fluorescent signal of GFP was observed 20 minutes after the puncture injury, but the strong fluorescent signal of GFP was not observed in the non-punctured GMO tobacco leaves (FIG. 9). To sum up, it is proved that the elements that regulate the functional genes in plants are injury-induced expression.

### Example 6. Identification of Secretory Characteristics of Inducible Secretory Expression Cassette and its Regulatory Elements

Signal peptides spAtPR1, spAtPR3, spAtPR5 and spAtPDF1.2 were added to the four expression cassettees of the functional gene GhJAZ8 respectively for inducible secretory expression cassette and its regulatory elements. In order to verify the functions of the signal peptides, GFP-GhJAZ8 protein and GFP-sp-GhJAZ8 protein subcellular positioning vectors were constructed. A GFP empty vector control was set (the GFP empty vector name is pBWA(V)HS-ccdb-Glosgfp, which is described in the literature “Liu F, Huang N, Wang L, Ling H, Sun T, Ahmad W, Muhammad K, Guo J, Xu L, Gao S, Que Y, Su Y. A Novel L-ascorbate Peroxidase 6 Gene, ScAPX6, Plays an Important Role in the Regulation of Response to Biotic and Abiotic Stresses in Sugarcane. Front Plant Sci. 2018 Jan. 17; 8:2262. doi: 10.3389/fpls.2017.02262. PMID: 29387074; PMCID: PMC5776131.”, the public can obtain this material from the applicant, and the obtained material can only be used to repeat this technical solution).

The fragment between BsaI and Eco31I (a small fragment between BsaI and Eco31I) of GFP empty vector (pBWA(V)HS-ccdb-Glosgfp) was replaced by DNA molecule whose nucleotide sequence is the 1164th-1520th position of SEQ ID No. 1, and other nucleotide sequences of GFP empty vector were kept unchanged, so as to obtain GFP-GhJAZ8 expression vector.

The fragment between BsaI and Eco31I (a small fragment between BsaI and Eco31I) of GFP empty vector (pBWA(V)HS-ccdb-Glosgfp) was replaced by DNA molecule whose nucleotide sequence was positions 1089-1520 in SEQ ID No. 1, and other nucleotide sequences of GFP empty vector were kept unchanged, thus obtaining the expression vector of GFP-spAtPR1-GhJAZ8.

The fragment between BsaI and Eco31I (a small fragment between BsaI and Eco31I) of GFP empty vector (pBWA(V)HS-ccdb-Glosgfp) was replaced by DNA molecule whose nucleotide sequence was positions 3345-3797 in SEQ ID No. 1, and other nucleotide sequences of GFP empty vector were kept unchanged, thus obtaining the expression vector of GFP-spAtPR3-GhJAZ8.

The fragment between BsaI and Eco31I (a small fragment between BsaI and Eco31I) of GFP empty vector (pBWA(V)HS-ccdb-Glosgfp) was replaced by DNA molecule whose nucleotide sequence was positions 5468-5890 in SEQ ID No. 1, and other nucleotide sequences of GFP empty vector were kept unchanged, so as to obtain the expression vector of GFP-spAtPR5-GhJAZ8.

The fragment between BsaI and Eco31I (a small fragment between BsaI and Eco31I) of GFP empty vector (pBWA(V)HS-ccdb-Glosgfp) was replaced by DNA molecule whose nucleotide sequence was positions 8260-8700 of SEQ ID No. 1, and other nucleotide sequences of GFP empty vector were kept unchanged, so as to obtain the expression vector of GFP-spAtPDF1.2-GhJAZ8.

The expression vectors obtained above were transiently transfected into rice protoplast cells, and the localization of GFP fluorescence in the cells was observed under laser confocal microscope.

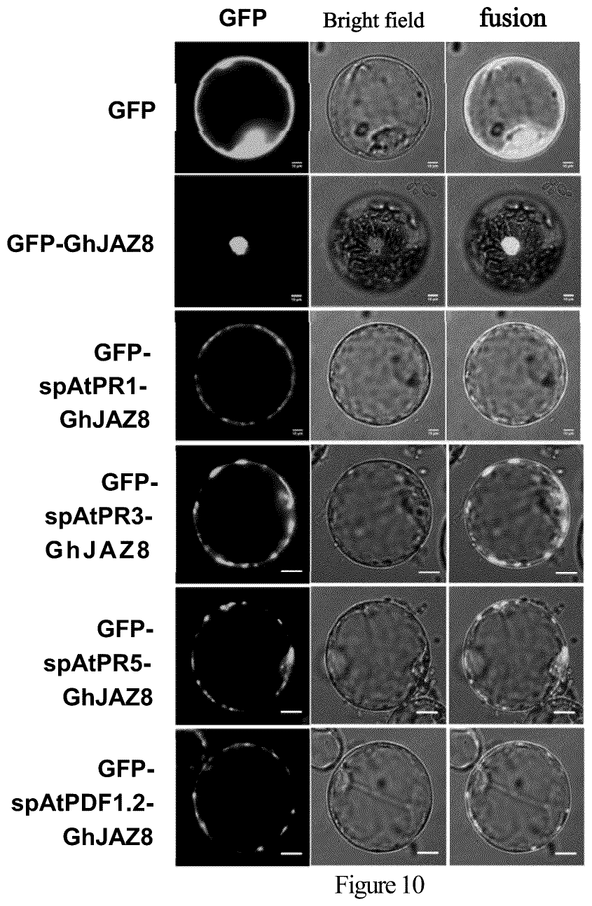

FIG. 10 shows the secretory evidence of inducible secretory expression cassette and its regulatory elements. In FIG. 10, GFP fluorescence of GFP empty vector control appears in multiple locations of cells, GhJAZ8-GFP fluorescence appears in the nucleus, GFP-spAtPR1-GhJAZ8, GFP-spAtPR3-GhJAZ8, GFP-spAtPR5-GhJAZ8, GFP-spAtPDF1.2-GhJAZ8 fluorescence appears between cells. Bright field, that is, no fluorescence excitation; fusion means excitation.

The results showed that the subcellular localization of the control GhJAZ8-GFP fusion protein without signal peptide was the nucleus of rice cell protoplast, while the subcellular localization of the sp-GhJAZ8-GFP fusion protein with signal peptide was between cells, that is, the original nuclear localization was changed after GhJAZ8 added secretory peptide sequence. GhJAZ8 protein is stored between cells, which is easier to be absorbed and ingested by pests when they bite. To sum up, it is proved that the elements that regulate the functional genes in plants are secretory.

The above description of the specific embodiments of the present disclosure does not limit the present disclosure, and those skilled in the art can make various changes or modifications according to the present disclosure, so long as they do not depart from the spirit of the present disclosure, which shall fall within the scope of the appended claims.

The present disclosure has been described in detail above. For those skilled in the art, the present disclosure can be implemented in a wide range with equivalent parameters, concentrations and conditions without departing from the spirit and scope of the present disclosure and without unnecessary experiments. Although the present disclosure has given specific embodiments, it should be understood that the present disclosure can be further improved. In a word, according to the principles of the present disclosure, this application is intended to include any changes, uses or improvements to the present disclosure, including changes made by conventional techniques known in the art, which depart from the disclosed scope in this application. According to the scope of the following appended claims, some basic features can be applied.

### INDUSTRIAL APPLICATION

The present disclosure realizes the damage-induced expression of functional genes for the first time, and verifies that the signal peptide can efficiently mediate the secretion and expression of foreign proteins. Before induction, the expression level of GFP reporter gene in transgenic leaves was very low, which indicated that the promoter of the plant recombinant expression vector containing inducible secretory expression cassette and its regulatory elements was low, and the promoter could start the expression of GFP reporter gene in large quantities during injury induction, which indicated that the promoter responded quickly and induced gene expression was high. In addition, the signal peptide guides the secretion of functional proteins between cells, which is very important for overexpressing toxic proteins in plants to improve plant resistance. These characteristics of the inducible secretory expression cassette and its regulatory elements meet the requirements of ideal regulatory elements in plant genetic engineering research, and can provide valuable materials for plant genetic engineering.

What is claimed is:

1. A DNA molecule, wherein the DNA molecule comprises four expression cassettes, and each expression cassette respectively comprises a promoter, a 5′UTR connected with the promoter, a signal peptide coding gene connected with the 5′UTR, a functional gene connected with the signal peptide coding gene, a 3′UTR connected with the functional gene and a terminator connected with the 3′UTR;
the promoter, the 5′UTR, the 3′UTR and the terminator are all derived from jasmonic acid-induced expression genes; the signal peptide is derived from secretory protein;
the nucleotide sequences of the functional genes in respective expression cassettes are different, and the nucleotide sequences of the promoter, the 5′UTR, the 3′UTR, the signal peptide coding gene and the terminator in each expression cassette are different or the same.

2. The DNA molecule according to claim 1, wherein the four expression cassettes are expression cassette A, expression cassette B, expression cassette C and expression cassette D;
the promoter of the expression cassette A is p-AtLOX2, and the nucleotide sequence of the p-AtLOX2 is the 1st-896th positions of SEQ ID No. 1; the 5′UTR of the expression cassette A is AtLOX2-5′UTR, and the nucleotide sequence of the AtLOX2-5′UTR is the 897th-1088th positions of SEQ ID No. 1; the 3′UTR of the expression cassette A is AtLOX2-3′UTR, and the nucleotide sequence of the AtLOX2-3′UTR is the 1548th-1672nd positions of SEQ ID No. 1; the terminator of the expression cassette a is t-AtLOX2, and the nucleotide sequence of the t-AtLOX2 is 1673rd-1872nd positions of SEQ ID No. 1;
the promoter of the expression cassette B is p-AtVSP2, and the nucleotide sequence of the p-AtVSP2 is the 1873rd-2964th positions of SEQ ID No. 1; the 5′UTR of the expression cassette B is AtVSP2-5′UTR, and the nucleotide sequence of the AtVSP2-5′UTR is the 2965th-3344th positions of SEQ ID No. 1; the 3′UTR of the expression cassette B is AtVSP2-3′UTR, and the nucleotide sequence of the AtVSP2-3′UTR is the 3829th-4078th positions of SEQ ID No. 1; the terminator of the expression cassette B is t-AtVSP2, and the nucleotide sequence of the t-AtVSP2 is the 4079th-4278th positions of SEQ ID No. 1;
the promoter of the expression cassette C is p-AtLOX3, and the nucleotide sequence of the p-AtLOX3 is 4279th-5278th positions of SEQ ID No. 1; the 5′UTR of the expression cassette C is AtLOX3-5′UTR, and the nucleotide sequence of the AtLOX3-5′UTR is the 5279th-5467th positions of SEQ ID No. 1; the 3′UTR of the expression cassette C is AtLOX3-3′UTR, and the nucleotide sequence of the AtLOX3-3′UTR is the 5892nd-6747th positions of SEQ ID No. 1; the terminator of the expression cassette C is t-AtLOX3, and the nucleotide sequence of the t-AtLOX3 is 6748th-6947th positions of SEQ ID No. 1;
the promoter of the expression cassette D is p-AtVSP1, and the nucleotide sequence of the p-AtVSP1 is the 6948th-7947th positions of SEQ ID No. 1; the 5′UTR of the expression cassette D is AtVSP1-5′UTR, and the nucleotide sequence of the AtVSP1-5′UTR is the 7948th-8259th positions of SEQ ID No. 1; the 3′UTR of the expression cassette D is AtVSP1-3′UTR, and the nucleotide sequence of the AtVSP1-3′UTR is the 9409th-9607th positions of SEQ ID No. 1; the terminator of the expression cassette D is t-AtVSP1, and the nucleotide sequence of the t-AtVSP1 is the 9608th-9807th positions of SEQ ID No. 1.

3. The DNA molecule according to claim 1, wherein the signal peptide is selected from sp-AtPR1, sp-AtPR3, sp-AtPR5 and sp-AtPDF1.2; the amino acid sequence of sp-AtPR1 is SEQ ID No.2, the amino acid sequence of sp-AtPR3 is SEQ ID No.3, the amino acid sequence of sp-AtPR5 is SEQ ID No.4, and the amino acid sequence of sp-AtPDF1.2 is SEQ ID No.5.

4. The DNA molecule according to claim 3, wherein the signal peptide coding gene is the coding gene of sp-AtPR1, sp-AtPR3, sp-AtPR5 or sp-AtPDF1.2, and the coding gene of sp-AtPRI is a DNA molecule with a nucleotide sequence of 1089th-1163rd positions of SEQ ID No. 1; the coding gene of sp-AtPR3 is a DNA molecule with a nucleotide sequence of 3345th-3440th positions of SEQ ID No. 1; the coding gene of sp-AtPR5 is a DNA molecule with a nucleotide sequence of 5468th-5533rd position of SEQ ID No. 1; the encoding gene of sp-AtPDF1.2 is a DNA molecule with the nucleotide sequence of 8260th-8343rd positions of SEQ ID No. 1.

5. A biomaterial, wherein the biomaterial is a recombinant vector, a recombinant microorganism, a transgenic plant cell line, a transgenic plant tissue or a transgenic plant organ containing the DNA molecule of claim 1.

6. A use, wherein the use is any one of A1)-A3):
A1) use of the DNA molecule of claim 1 in damage-induced expression of plant functional genes;
A2) use of the DNA molecule according to claim 1 in improving the expression of plant functional genes; and
A3) use of the DNA molecule according to claim 1 in plant stress resistance.

7. Use, wherein the use is any one of B1)-B3):
B1) use of the biomaterial according to claim 5 in damage-induced expression of plant functional genes;
B2) use of the biomaterial according to claim 5 in improving the expression of plant functional genes; and
B3) use of the biomaterial according to claim 5 in plant stress resistance.

8. A method for improving plant stress resistance, wherein the method comprises expressing a functional gene in a recipient plant with the DNA molecule according to claim 1.

9. The method according to claim 8, wherein the functional gene is a damage-induced gene.
